(19) United States
(12) Patent Application Publication
(10) Pub. No.: US 2012/0184751 A1
(43) Pub. Date: Jul. 19, 2012

(54) POLYMORPHIC FORM OF OLMESARTAN MEDOXOMIL

(75) Inventors: Ashwini Kumar Kapoor, Aurangabad (IN); Hiten Sharadchandra Mehta, Ahmedabad (IN); Asok Nath, Gurgaon (IN); Mohan Prasad, Gurgaon (IN)

(73) Assignee: RANBAXY LABORATORIES LIMITED, New Delhi, Delhi (IN)

(21) Appl. No.: 13/388,091

(22) PCT Filed: Jul. 29, 2010
(86) PCT No.: PCT/IB10/53463

§ 371 Date: Apr. 10, 2012

(30) Foreign Application Priority Data
Jul. 31, 2009 (IN) 1591/DEL/2009

(51) Int. Cl. C07D 405/14

(52) U.S. Cl. 548/253

(57) ABSTRACT

The present invention relates to a polymorphic form of olmesartan medoxomil and a process for the preparation of crystalline olmesartan medoxomil.

### FIELD OF THE INVENTION

The present invention relates to a polymorphic form of olmesartan medoxomil and a process for the preparation of crystalline olmesartan medoxomil.

### BACKGROUND OF THE INVENTION

Olmesartan medoxomil is chemically 2,3-dihydroxy-2-butenyl-4-(1-hydroxy-1-methylethyl)-2-propyl-1-[p-(o-1H-tetrazol-5-ylphenyl)benzyl]imidazole-5-carboxylate, cyclic 2,3-carbonate, having the structure of Formula I.

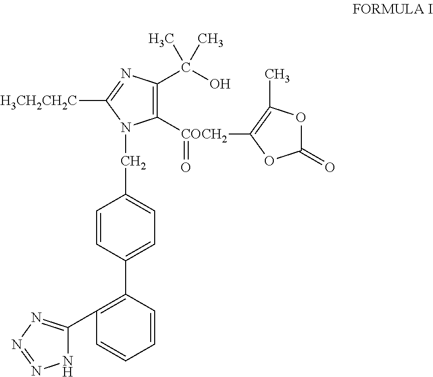

Olmesartan medoxomil is a prodrug, which is hydrolyzed to olmesartan during absorption from the gastrointestinal tract. Olmesartan is a selective AT1 subtype angiotensin II receptor antagonist and useful for the treatment of hypertension.

U.S. Pat. No. 5,616,599 describes a process for the preparation olmesartan medoxomil, wherein the residue obtained after reaction is purified by column chromatography through silica gel using 1:9 and 1:4 by volume mixtures of methanol and methylene chloride as eluent to give olmesartan medoxomil melting at 170° to 172° C. U.S. Pat. No. 5,616,599 also describes a process, wherein the residue obtained after reaction is crystallized in ethyl acetate to give olmesartan medoxomil as crystals melting at 177° to 180° C.

U.S. Publication No. 2006/0281800 describes a process for the preparation of polymorph form G of the olmesartan medoxomil. According to the process described in the above application, olmesartan medoxomil is added slowly added into methanol. The suspension is slowly heated to a temperature ranging from about 45° to about 50° C., maintained for about 60 minutes and about 60-70 ml of methanol is distilled. The mixture is then slowly cooled to room temperature and further cooled to a temperature ranging from about 0° to about 5° C. such that crystalline olmesartan medoxomil is precipitated out of solution. The precipitated product is filtered, washed with methanol and dried to obtain polymorph form G of olmesartan medoxomil.

EP Application No. 1,801,111 A1 describes a process for the preparation of olmesartan medoxomil hemihydrate. According to the process described in the above application, olmesartan medoxomil is dissolved in dimethylsulfoxide, water is added to the solution and stirred for several hours to obtain a crystalline suspension, which is dried in a vacuum to obtain olmesartan medoxomil hemihydrates.

EP Application No. 1,801,111 A1 also describes processes for the preparation of amorphous or partially crystalline olmesartan medoxomil by vacuum evaporation of olmesartan medoxomil solutions in methanol, ethanol, propanol or acetonitrile. The initial solutions are prepared either by heating at reflux temperature or by using high dilution of solvent such as 1 g/100 ml. According to EP Application No. 1,801,111 A1, when amorphous olmesartan medoxomil is 75% relative humidity at 40° C. for one month it converts into hydrated olmesartan medoxomil having crystalline appearance.

PCT Publication No. WO 2008/149160 describes processes for the preparation of amorphous olmesartan medoxomil by leaving the open crystallization dish containing olmesartan medoxomil solutions in methanol, ethanol or acetonitrile in a fume-hood overnight. The initial solutions are prepared by ultrasonication at room temperature.

PCT Publication No. WO 2008/149155 describes processes for the preparation of olmesartan medoxomil crystalline form B by use of water or cyclohexane as an anti-solvent from tetrahydrofuran solutions of olmesartan medoxomil, by use of cyclohexane as anti-solvent from acetone solutions of olmesartan medoxomil or by use of cyclohexane as anti-solvent from dichloromethane solutions of olmesartan medoxomil. The initial solutions are prepared by ultrasonication at room temperature. The XRPD (X-ray Powder Diffractogram) pattern of olmesartan medoxomil crystalline form B exhibits a halo along with crystalline peaks. PCT Publication No. WO 2008/149155 says that the increased background in the range of 35-40° 2θ in the XRPD of crystalline form B is due to the XRPD sample holder.

The preparation of crystalline olmesartan medoxomil requires a first step of dissolving olmesartan medoxomil in a solvent while it has limited solubility in the solvents used in the above prior art references. The prior art methods employ heating, ultrasonication or high dilution to achieve dissolution of olmesartan medoxomil and yield amorphous or partially crystalline olmesartan medoxomil.

### BRIEF DESCRIPTION OF THE DRAWINGS

FIG. 1 depicts X-Ray Powder Diffractogram (XRPD) of polymorphic form R of olmesartan medoxomil.

FIG. 1A provides table of the XRPD (FIG. 1) of polymorphic form R of olmesartan medoxomil.

FIG. 2 depicts Fourier-Transform Infra-red (FTIR) spectrum of polymorphic form R of olmesartan medoxomil.

FIG. 3 depicts Differential Scanning Calorimetry (DSC) thermogram of polymorphic form R of olmesartan medoxomil.

FIG. 4 depicts Thermogravimetric Analysis (TGA) of polymorphic form R of olmesartan medoxomil.

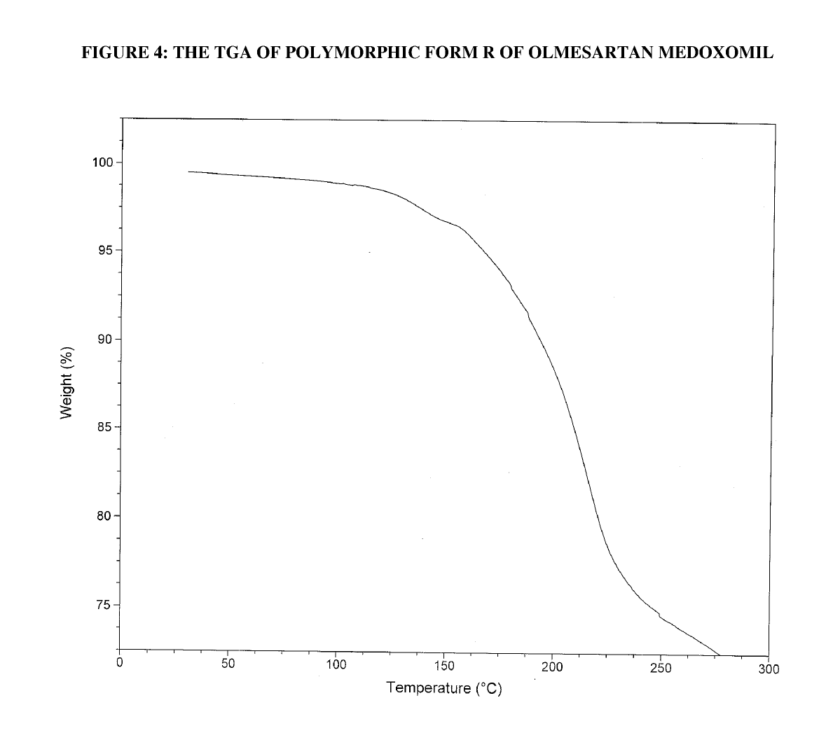

FIG. 5 depicts X-Ray Powder Diffractogram (XRPD) of crystalline olmesartan medoxomil obtained according to the reference example.

FIG. 5A provides table of the XRPD (FIG. 5) of crystalline olmesartan medoxomil obtained according to the reference example.

FIG. 6 depicts Fourier-Transform Infra-red (FTIR) spectrum of crystalline olmesartan medoxomil obtained according to the reference example.

FIG. 7 depicts Differential Scanning Calorimetry (DSC) thermogram of crystalline olmesartan medoxomil obtained according to the reference example.

### SUMMARY OF THE INVENTION

The present inventors have found that crystalline olmesartan medoxomil can be prepared without any need for heating, ultrasonication or high dilution. The present method also provides a way to prepare crystalline olmesartan medoxomil from various solvents by employing conventional isolation methods without impacting the polymorphic integrity.

The present inventors have also observed that the process described herein provides a polymorphic form of olmesartan medoxomil which is characteristically different from the forms described elsewhere, including references mentioned above. The polymorphic form of the present invention is designated as polymorphic form R of olmesartan medoxomil. Form R of olmesartan medoxomil is stable, reproducible and suitable for developing pharmaceutical dosage forms.

### DETAILED DESCRIPTION OF THE INVENTION

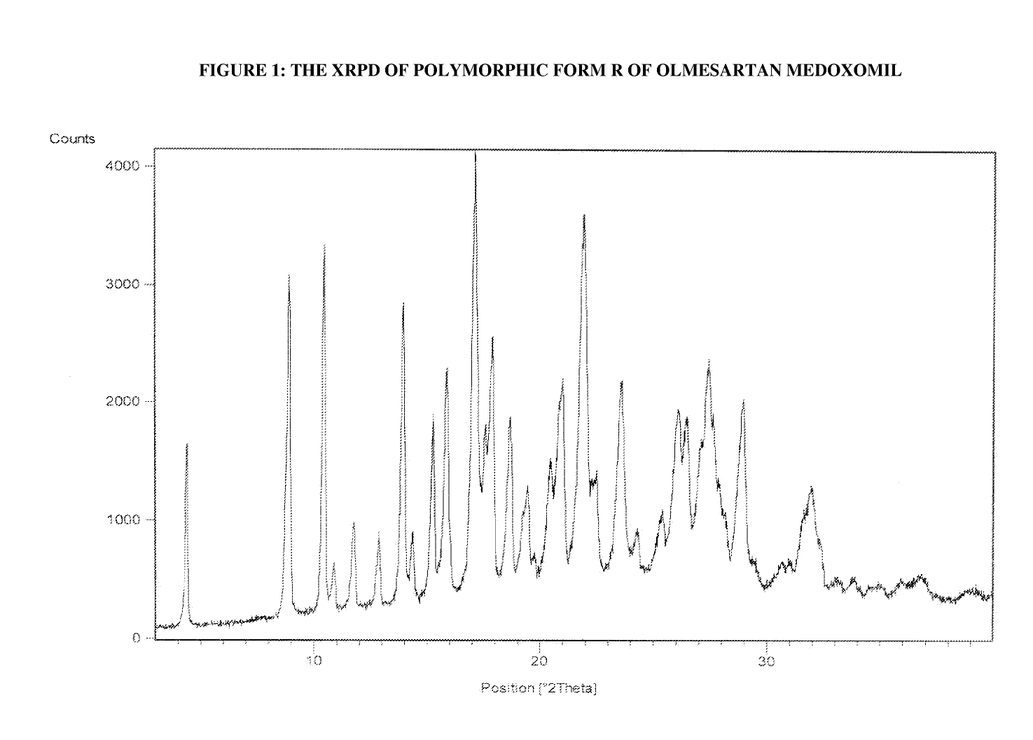

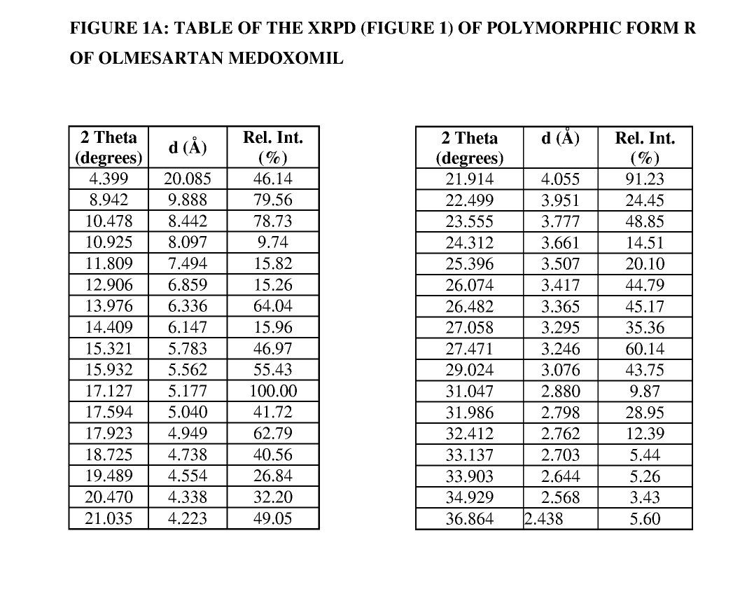

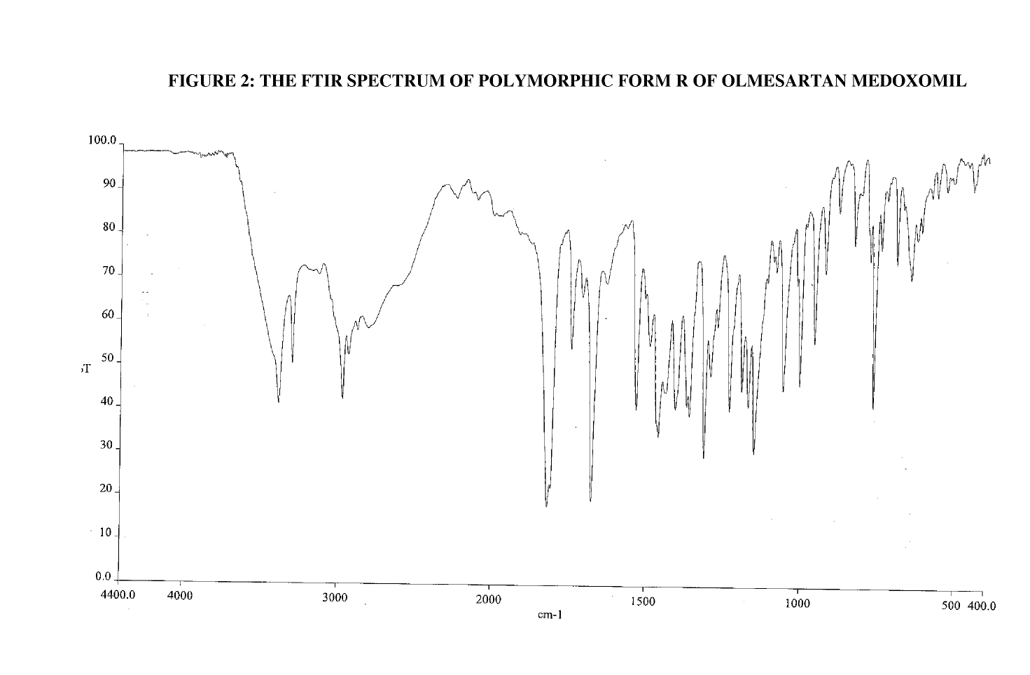

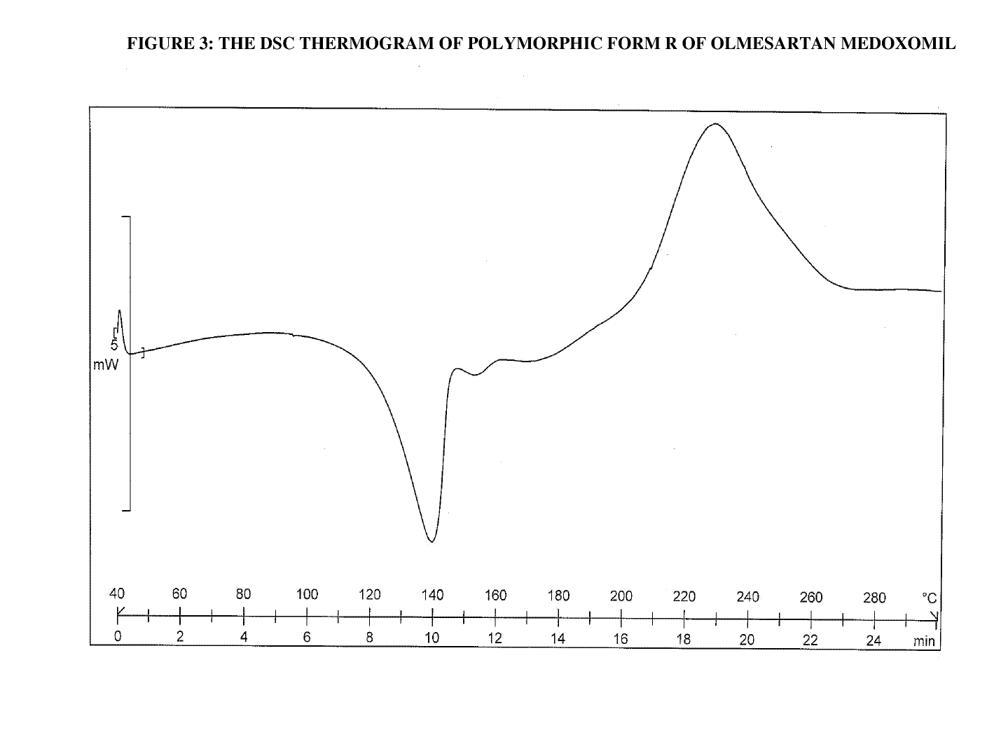

A first aspect of the present invention provides polymorphic form R of olmesartan medoxomil. The polymorphic form R of olmesartan medoxomil has substantially the same XRPD pattern as depicted in FIG. 1. The XRPD pattern of the polymorphic form R of olmesartan medoxomil shows characteristic d-spacing [Å] values substantially at 2.4, 2.6, 2.6, 2.7, 2.8, 2.8, 2.9, 3.1, 3.2, 3.3, 3.4, 3.4, 3.5, 3.7, 3.8, 3.9, 4.0, 4.2, 4.3, 4.6, 4.7, 4.9, 5.0, 5.2, 5.6, 5.8, 6.1, 6.3, 6.9, 7.5, 8.1, 8.4, 9.9 and 20.1. The polymorphic form R of olmesartan medoxomil has substantially the same FTIR pattern as depicted in FIG. 2. The polymorphic form R of olmesartan medoxomil has substantially the same DSC pattern as depicted in FIG. 3. The DSC of the polymorphic form R of olmesartan medoxomil exhibits two melting endotherms between about 128° and about 148° C. and between about 153° to about 165° C. and a broad exotherm between about 204° to about 270° C.

A second aspect of the present invention provides a process for the preparation of crystalline olmesartan medoxomil, wherein the process comprises,
(a) dissolving olmesartan medoxomil in an organic solvent at a temperature of about 30° C. or below in the presence of a base,
(b) stiffing the solution obtained in step a) for a sufficient time to obtain a solid, and
(c) isolating crystalline olmesartan medoxomil from the mixture thereof.

The starting olmesartan medoxomil can be prepared according to the methods described in the prior art, for example, U.S. Pat. No. 5,616,599. The olmesartan medoxomil is dissolved in an organic solvent at a temperature of about 30° C. or below in the presence of a base. The temperature for dissolving olmesartan medoxomil may be, for example, from about 15° C. to about 30° C. The dissolution may be performed, for example, by suspending olmesartan medoxomil in an organic solvent, followed by treating the suspension with a base, or by contacting olmesartan medoxomil with an organic solvent pre-treated with a base. The base is used in a quantity sufficient to dissolve olmesartan medoxomil in the organic solvent.

The organic solvent may be selected from the group comprising of esters, ketones, halogenated hydrocarbons, alcohols, nitriles and mixtures thereof. The examples of ketones include acetone, 2-pentanone, 3-pentanone, methylisobutyl ketone, methyl ethyl ketone, cyclopentanone, cyclohexanone and the like. The examples of esters include ethylacetate, ethyl propionate, ethyl butanoate and the like. The examples of halogenated hydrocarbons include methylene chloride, ethylene dichloride, chloroform, and the like. The examples of alcohols include methanol, ethanol, isopropanol, isobutyl alcohol and the like. The examples of nitriles include acetonitrile, propionitrile, butanenitrile and the like.

The base may be organic or inorganic. The base may be, for example, ammonia, ammonium hydroxide or aqueous ammonia, ammonium bicarbonate, ammonium carbonate, alkali metal or alkaline earth metal carbonate or bicarbonate, methylamine, ethylamine, propylamine, morpholine, pyridine or a mixture thereof.

The solution so obtained is stirred for a sufficient time to obtain a solid. The stirring may be carried out for about 10 minutes to about 10 hours, for example 20 minutes to about 1 hour. The solid obtained is isolated from the mixture by conventional methods, for example, filtration, solvent evaporation, decantation, or a combination thereof, to obtain crystalline olmesartan medoxomil. The crystalline olmesartan medoxomil is isolated, for example, as polymorphic form R of olmesartan medoxomil described in the previous aspect of the invention.

A third aspect of the present invention provides a pharmaceutical composition comprising polymorphic form R of olmesartan medoxomil and a pharmaceutically acceptable carrier.

A fourth aspect of the present invention provides a method of treating hypertension comprising administering to a subject in need of such treatment a therapeutically effective amount of polymorphic form R of olmesartan medoxomil.

The XRPD was determined by using Panalytical X'Pert Pro X-Ray Powder Diffractometer in the range 3° to 40° 2θ and under tube voltage and current of 45 Kv and 40 mA respectively. Copper radiation of wavelength 1.54 angstrom and Xceletor detector was used.

The TGA was recorded on TA(Q500) (Rate of heating=10° C./minute). The DSC was recorded on Mettler Toledo(DSC 821) (Rate of heating=10° C./minute). The FTIR was recorded in KBr on Perkin Elmer FTIR (Spectrum One) instrument.

In the following section embodiments are described by way of examples to illustrate the process of invention. However, these are not intended in any to limit the scope of present invention. Several variants of these examples would be evident to persons ordinarily skilled in the art.

### EXAMPLES

### Example 1

### Preparation of Crystalline Olmesartan Medoxomil (Polymorphic Form R):

Ammonia solution (25%; 2 ml) was added to a suspension of olmesartan medoxomil (10 g) in acetonitrile (50 ml) to dissolve the solid at 25° to 30° C. The solution was stirred at 25° to 30° C. and the separation of the solid was observed. The suspension was further stirred for 30 minutes, filtered and washed with acetonitrile (2×10 ml) and dried under vacuum at 50° to 60° C. to obtain the title product.

### Yield: 90%

### Example 2

### Preparation of Crystalline Olmesartan Medoxomil (Polymorphic Form R):

Ammonia solution (25%; 2 ml) was added to a suspension of olmesartan medoxomil (10 g) in acetone (50 ml) to dissolve the solid at 25° to 30° C. The solution was stirred at 25° to 30° C. and the separation of the solid was observed. The suspension was further stirred for 30 minutes, filtered and washed with acetone (2×10 ml) and dried under vacuum at 50° to 60° C. to obtain the title product.

### Yield: 83.5%

HPLC purity: 98%

### Example 3

### Preparation of Crystalline Olmesartan Medoxomil (Polymorphic Form R):

Ammonia solution (25%; 2 ml) was added to a suspension of olmesartan medoxomil (10 g) in ethyl acetate (50 ml) to dissolve the solid at 25° to 30° C. The solution was stirred at 25° to 30° C. and the separation of the solid was observed. The suspension was further stirred for 30 minutes, filtered and washed with ethyl acetate (2×10 ml) and dried under vacuum at 50° to 60° C. to obtain the title product.

### Yield: 95%

HPLC purity: 98.56%

### Reference Example

### Preparation of Crystalline Olmesartan Medoxomil

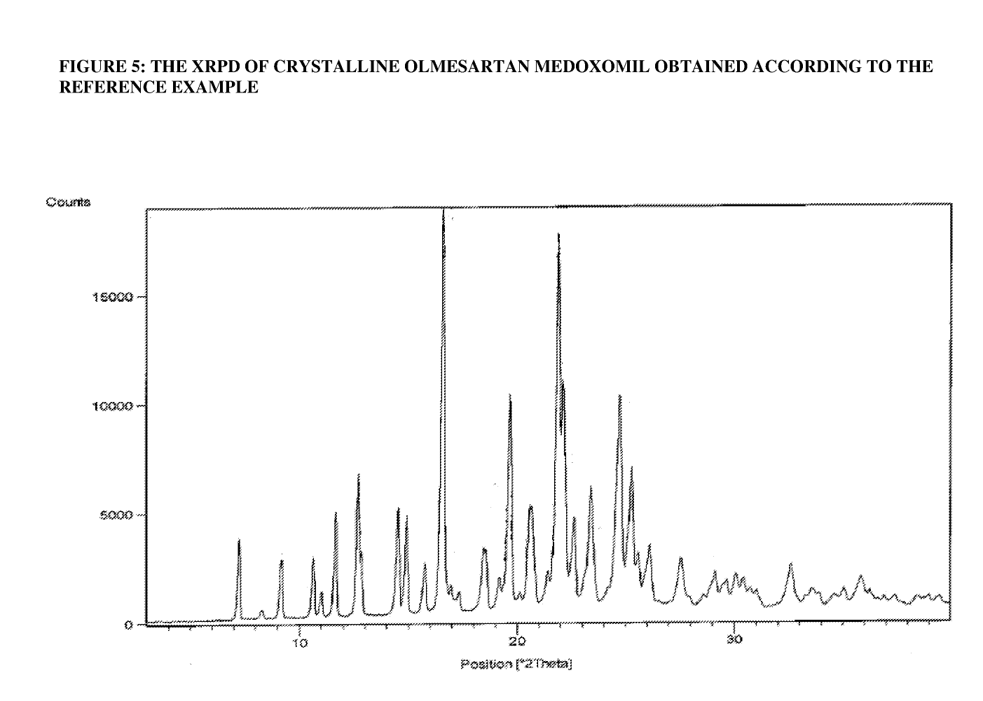

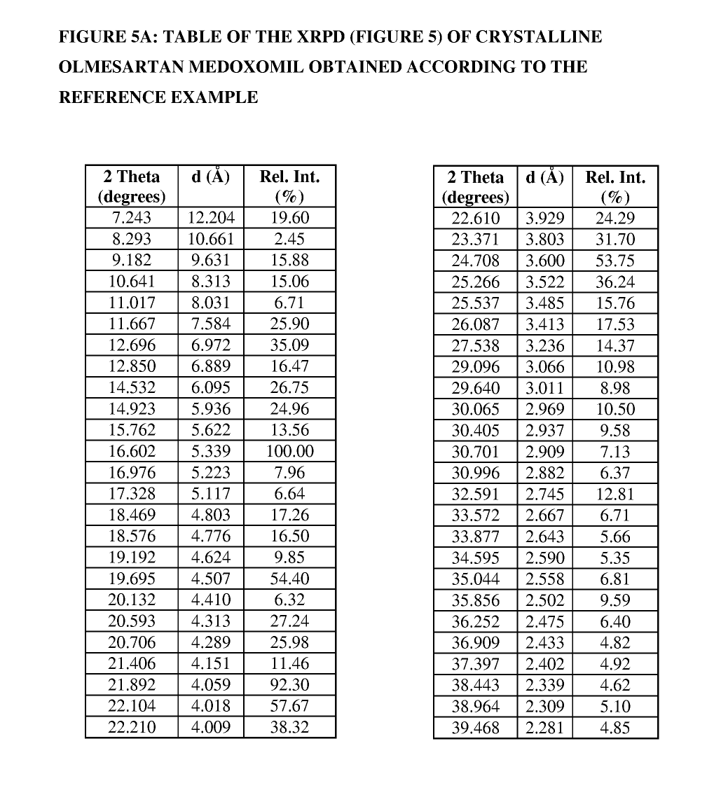

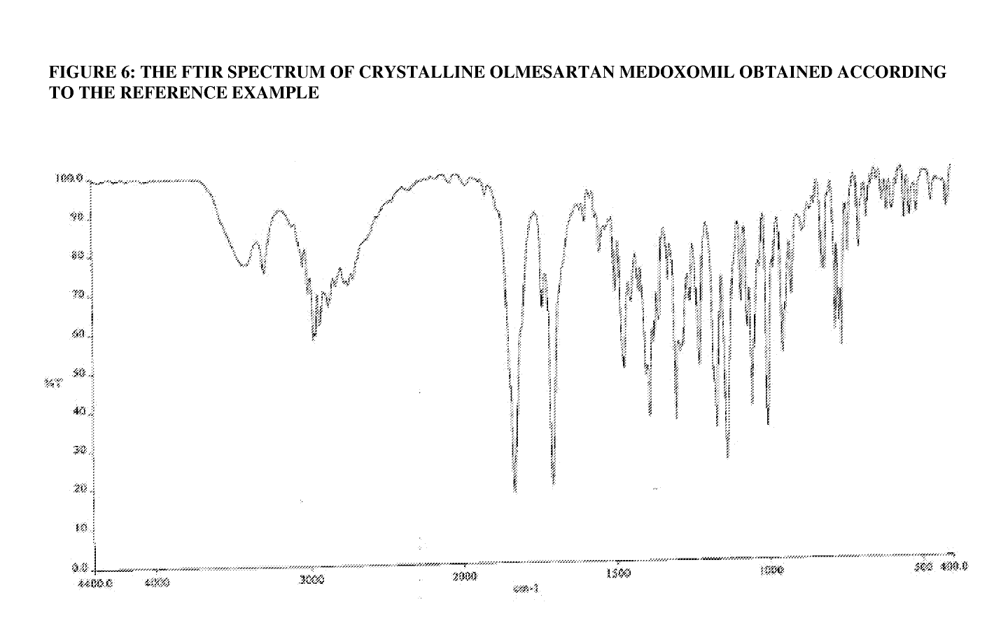

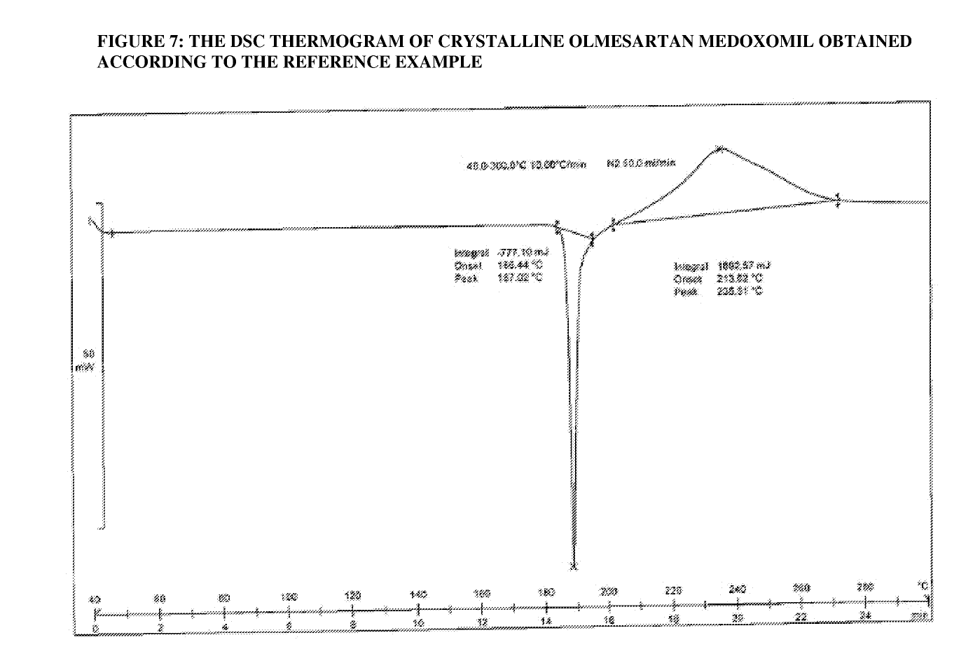

Olmesartan medoxomil (2 g) was dissolved in ethyl acetate (100 ml) by heating at 60° to 65° C. The solution was cooled to 40° to 45° C. and filtered at the same temperature. The filtrate was cooled slowly to 25° to 30° C. The solid obtained was filtered and dried under vacuum at 40° C. to obtain the title product having the XRPD, FTIR and DSC as depicted in FIGS. 5, 6 and 7 respectively.

We claim:

1. Polymorphic form R of olmesartan medoxomil having substantially the same XRPD pattern as depicted in FIG. 1.

2. Polymorphic form R of olmesartan medoxomil showing characteristic d-spacing [Å] values substantially at 2.4, 2.6, 2.6, 2.7, 2.8, 2.8, 2.9, 3.1, 3.2, 3.3, 3.4, 3.4, 3.5, 3.7, 3.8, 3.9, 4.0, 4.2, 4.3, 4.6, 4.7, 4.9, 5.0, 5.2, 5.6, 5.8, 6.1, 6.3, 6.9, 7.5, 8.1, 8.4, 9.9 and 20.1. in the XRP diffractogram

3. Polymorphic form R of olmesartan medoxomil having substantially the same FTIR pattern as depicted in FIG. 2.

4. Polymorphic form R of olmesartan medoxomil having substantially the same DSC pattern as depicted in FIG. 3.

5. Polymorphic form R of olmesartan medoxomil exhibiting two melting endotherms between about 128° and about 148° C. and between about 153° to about 165° C. and a broad exotherm between about 204° to about 270° C. in the DSC.

6. A process for the preparation of crystalline olmesartan medoxomil, wherein the process comprises:
(a) dissolving olmesartan medoxomil in an organic solvent at a temperature of about 30° C. or below in the presence of a base,
(b) stirring the solution obtained in step a) for a sufficient time to obtain a solid, and
(c) isolating crystalline olmesartan medoxomil from the mixture thereof.

7. A process according to claim 6, wherein the temperature for dissolving olmesartan medoxomil is from about 15° C. to about 30° C.

8. A process according to claim 6, wherein the base is used in a quantity sufficient to dissolve olmesartan medoxomil.

9. A process according to claim 6, wherein the organic solvent is selected from a group comprising of esters, ketones, halogenated hydrocarbons, alcohols, nitriles and mixtures thereof.

10. A process according to claim 6, wherein the base is ammonia, ammonium hydroxide or aqueous ammonia, ammonium bicarbonate, ammonium carbonate, alkali metal or alkaline earth metal carbonate or bicarbonate, methylamine, ethylamine, propylamine, morpholine, pyridine or a mixture thereof.

11. A process according to claim 6, wherein the stiffing is carried out for about 10 minutes to about 10 hours.

12. A process according to claim 6, wherein the crystalline olmesartan medoxomil is polymorphic form R of olmesartan medoxomil.
